(19) United States
(12) Patent Application Publication
(10) Pub. No.: US 2023/0097960 A1
(43) Pub. Date: Mar. 30, 2023

(54) VITRO DEVICE TO MEASURE STOOL ALKALINE PHOSPHATEASE

(71) Applicant: MADHU S. MALO, BURLINGTON, MA (US)

(72) Inventor: MADHU S. MALO, BURLINGTON, MA (US)

(21) Appl. No.: 17/300,690

(22) Filed: Sep. 29, 2021

(51) Int. Cl. C12Q 1/42

(52) U.S. Cl. CPC C12Q 1/42

(57) ABSTRACT

This invention describes a de novo in vitro device to measure STAP. Measurement of stool alkaline phosphatase (STAP) will be pivotal in determining the physiological as well as pharmacological effects of intestinal alkaline phosphatase (IAP), the major component of STAP. The device is described for measuring phosphatase concentration in stool. The device (chromogenic STAP Test) allows persistent contact of a stool sample for a specific period of time (e.g., 30 min) with a piece of chromatography paper (strip) impregnated with a STAP substrate (p-nitrophenyl phosphate, p-NPP), and then the developed color (yellow) is compared with standards thus providing the STAP concentration. For a permanent record, the developed color along with standards is photographed.

### BACKGROUND

Alkaline phosphatases (APs, E.C.3.I.3.1.) are membrane-bound glycoproteins that optimally catalyze the hydrolysis of phosphate monoesters at high pH with the release of inorganic phosphate (Millan, 2005, https://www.ncbi.nlm.nih.gov/pmc/articles/PMC2254479/; Sharma et al, 2014, https://www.ncbi.nlm.nih.gov/pmc/articles/PMC4062654/). Most prokaryotes and eukaryotes produce APs. Functionally active mammalian APs are homodimers, and each catalytic site contains three metal ions (two Zn⁺ and one Mg⁺) necessary for enzymatic activity (Millan, 2005, https://www.ncbi.nlm.nih.gov/pmc/articles/PMC2254479/). Various isoforms of mammalian APs exist, namely intestinal alkaline phosphatase (IAP), placental AP, tissue nonspecific AP (liver/bone/kidney/neutrophils AP, TNAP), and germ cell AP (Kaliannan et al., 2013, https://www.ncbi.nlm.nih.gov/pubmed/23569246). The AP isoforms share significant structural homology as well as functional similarities.

Intestinal alkaline phosphatase (IAP) is exclusively expressed in villus-associated enterocytes of proximal small intestine, and bidirectionally secreted into the intestinal lumen as well as the systemic circulation (Eliakim et al., 1991, https://www.ncbi.nlm.nih.gov/pubmed/1671644). IAP travels downwards from the proximal small intestine to the distal large intestine and then excreted with stool (Malo et al., 2010, https://www.ncbi.nlm.nih.gov/pubmed/20947883). Stool alkaline phosphatase (STAP) is composed of approximately 80% LAP, and 20% bacterial alkaline phosphatase as TNAP concentration is very low (Malo, 2015, https://www.ncbi.nlm.nih.gov/pubmed/26844282).

Physiologically, IAP maintains intestinal bacterial homeostasis and gut mucosal integrity, detoxifies bacterial toxins, and limits fat absorption (Malo, 2015, https://www.ncbi.nlm.nih.gov/pubmed/26844282). The deficiency of IAP leads to the development of diabetes and dyslipidemia in mice (Kaliannan et al., 2013, https://www.ncbi.nlm.nih.gov/pubmed/23569246), and LAP deficiency is associated with diabetes in humans (Malo, 2015, https://www.ncbi.nlm.nih.gov/pubmed/26844282). IAP deficiency is also associated with ischemic heart disease (Malo et al., 2019, https://pubmed.ncbi.nlm.nih.gov/31915470/).

Regulation and function of LAP have been extensively reviewed (Lalles, 2014, https://www.ncbi.nlm.nih.gov/pubmed/24506153; Estaki et al., 2014, https://www.ncbi.nlm.nih.gov/pubmed/25400448; Sharma et al, 2014, https://www.ncbi.nlm.nih.gov/pmc/articles/PMC4062654/); Bucket et al., 2013, https://www.ncbi.nlm.nih.gov/pubmed/23860646; Vaishnava and Hooper, 2007, https://www.ncbi.nlm.nih.gov/pubmed/18078687).

Pharmacologically, oral IAP supplementation prevents antibiotic-induced susceptibility to enteric pathogens such as Salmonella typhimurium and Clostridium difficile (Malo et al., 2010, https://www.ncbi.nlm.nih.gov/pubmed/20947883; Alam et al., 2014, https://www.ncbi.nlm.nih.gov/pubmed/23598380). Further, oral IAP supplementation not only prevents but also cures the high fat diet-induced metabolic syndrome in mice (Kaliannan et al., 2013, https://www.ncbi.nlm.nih.gov/pubmed/23569246). IAP supplementation have been shown to have beneficial effects against colitis, peritonitis, and acute kidney injury in humans and mice (Malo, 2015, https://www.ncbi.nlm.nih.gov/pubmed/26844282).

Determination of STAP concentration might contribute to our understanding of physiological and pharmacological effects of IAP. Recently, a biochemical assay to determine STAP has been reported (Malo, 2015, https://www.ncbi.nlm.nih.gov/pubmed/26844282), however, no home-based STAP assay (STAP Test) has yet been developed.

### SUMMARY OF THE INVENTION

According to the disclosure, a home-based de novo in vitro device for measuring stool alkaline phosphatase (STAP) has been developed. The device (chromogenic STAP Test) allows persistent contact of a stool sample for a specific period of time (e.g., 30 min) with a piece of chromatography paper (strip) impregnated with a STAP substrate (p-nitrophenyl phosphate, p-NPP), and then the developed color (yellow) is compared with standards thus providing the STAP concentration. For a permanent record, the developed color along with standards is photographed.

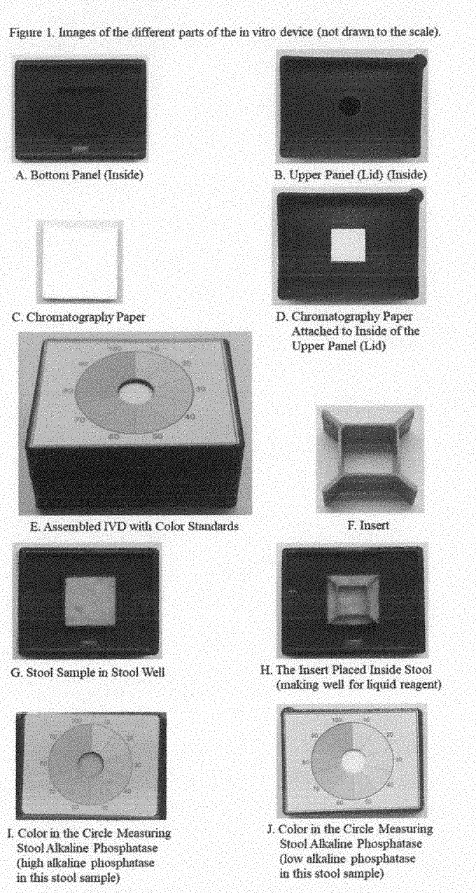

As a modification of the device, the chromatography paper is not impregnated with STAP substrate, wherein the substrate and buffer are provided in different containers. An Insert (FIG. 1F, see below) is placed deep inside stool thus making a well and the mixture of substrate and buffer is poured in the insert well. The mixture is kept for a short period of time (usually 3 minutes), the upper lid is placed and then the device is inverted thus transferring the developed color to the chromatography paper followed by STAP quantitation and photography.

### BRIEF DESCRIPTION OF THE DRAWINGS

FIG. 1 showing the images of different parts of the in vitro device (not drawn to the scale).
A. Bottom Panel (Inside)
B. Upper Panel (Lid) (Inside)
C. Chromatography Paper
D. Chromatography Paper Attached to Inside of the Upper Panel (Lid)
E. Assembled IVD with Color Standards
F. Insert
G. Stool Sample in Stool Well
H. The Insert Placed Inside Stool (making well for liquid reagent)
I. Color in the Circle Measuring Stool Alkaline Phosphatase (high alkaline phosphatase in this stool sample)
J. Color in the Circle Measuring Stool Alkaline Phosphatase (low alkaline phosphatase in this stool sample)

### DETAILED DESCRIPTION

Measurement of stool alkaline phosphatase (STAP) will be pivotal in determining the physiological as well as pharmacological effects of intestinal alkaline phosphatase (IAP), the major component of STAP. This invention describes a de novo in vitro device to measure STAP. In principle, a piece of chromatography paper impregnated with a STAP substrate (e.g., p-nitrophenyl phosphate, p-NPP) is allowed to remain constantly in contact with a stool sample for a certain period of time (e.g., 30 min). STAP in the stool sample reacts with the p-NPP substrate and produces p-nitrophenol (p-NP) and inorganic phosphate. The p-NP solution is yellow in color (p-NPP solution is clear) and it is absorbed by the chromatography paper rendering the paper yellow, the intensity of which is compared with the standards thus determining the concentration of STAP. Optionally, for future reference, a photograph is taken. The STAP concentration is expressed as U/gm stool.

### Development of STAP Substrate Strip

A sheet of chromatography paper is soaked in the STAP substrate solution (1.25 M diethanolamine (DEA) buffer, pH 10.2, 0.6 mM magnesium chloride, 10 mM p-NPP). The paper is then dried in a dark room. The dried paper is cut into pieces (strips) to attach in the top panel (lid) of the in vitro device (IVD, see below). The strip is then glued to the lid.

### STAP Assay with a Biochemistry Analyzer

STAP assay with a biochemistry analyzer has been previously described (Malo, 2015; https://www.ncbi.nlm.nih.gov/pubmed/26844282). The biochemistry analyzer was just used in this case to develop the standards for the IVD.

Homogenization of stool: The supernatant of a homogenized stool suspension was used for STAP assay. A small amount of stool (milligrams) was measured and then the ‘stool dilution buffer’ (10 mM (millimolar) Tris-HCl, pH 8.0, 1 mM magnesium chloride, 10 μM (micromolar) zinc chloride) was added at a defined ratio. Briefly, 5 ml (milliliters) of stool dilution buffer was added to 100 mg of stool. The sample was vigorously vortexed to prepare a homogenized stool suspension. This suspension was then centrifuged at 10,000×g for 20 min, and the supernatant containing IAP was collected and assayed for IAP concentration.

Alkaline Phosphatase Assay using a Biochemistry Analyzer: The stool supernatant was assayed for alkaline phosphatase (AP) following an established protocol using a commercially available biochemical assay kit (Linear Chemicals S.L., Barcelona, Spain) and an automatic biochemistry analyzer (Sinnowa Medical Science & Technology Co., Ltd, Nanjing, Jiangsu, China; Model: Sinnolab MT 5000, Version 5.00). In brief, 20 μl of supernatant were added to 1 ml of enzyme assay buffer (1.25 M diethanolamine (DEA) buffer, pH 10.2, 0.6 mM magnesium chloride) containing 10 mM p-nitrophenyl phosphate (p-NPP), and the reaction mixture was incubated for one min at 37° C. This was followed by measuring the AP concentration by the analyzer pre-calibrated with the AP standards. It is important to note that most (approx. 80%) of the AP activity in stool is due to IAP and the rest is due to bacterial AP as TNAP is very low in stool (Malo, 2015, https://www.ncbi.nlm.nih.gov/pubmed/26844282). Accordingly, the stool AP values are expressed as units of IAP/gm stool.

### Development of Standards

To develop the standards of the home-based STAP test IVD, a stool sample was assayed for STAP concentration using a biochemistry analyzer. The same stool sample was then applied to the home-based IVD allowing for the development of the yellow color in the chromatography paper (see below). After a specific period of time (e.g., 30 min) the chromatography paper in the IVD turned yellow, and this yellow color was photographed and assigned as the standard in the IVD for the same STAP concentration as determined by the biochemistry analyzer. Stool samples with various concentrations of STAP were assayed both by biochemistry analyzer as well as by the IVD. Different photographs with different intensities of the yellow color thus obtained were used to compile a series of standards ranging from 0-100 U of STAP per gm stool.

### Validation of the Home-Based STAP Test IVD

The same stool sample was assayed for STAP activity using a biochemistry analyzer as well as by the home-based STAP test IVD (this invention). STAP concentration was determined by the biochemistry analyzer as described above. For measuring STAP concentration by the IVD, the fresh stool sample was used to fill-in the wells of the STAP test device (bottom panel), and the surface of stool was made smooth using a spatula. The lid (top panel of the IVD) containing the dry piece of chromatography paper impregnated with STAP substrate (p-NPP) was made wet by submersing the lid in water for a few seconds. Excess water in the lid was discarded by gentle shaking and absorbing the remaining water with a piece of tissue paper. The lid was then placed atop the bottom panel ensuring that the stool and the chromatography paper containing the STAP substrate (p-NPP) were in contact. After a certain time period (e.g., 30 min) the color (yellow) developed and STAP concentration was determined comparing the intensity of the yellow color with the standards developed via biochemistry analysis. The process was repeated using multiple stool samples with different STAP concentrations which were assayed by the biochemistry analyzer as well as by the home-based STAP test device in order to validate the results of the IVD (see examples below).

### EXAMPLES

### Example 1. STAP Activity Determined by the Home-Based STAP Test Device is Similar to STAP Activity Determined by a Biochemistry Analyzer.

| | STAP Value Determined by | STAP Value Determined by |
|---|---|---|
| | Biochemistry Analyzer | Home-Based STAP Test |
| Stool Sample | (U/gm Stool) | (U/gm Stool) |
| Sample 1 | 96 | 90 |
| Sample 2 | 77 | 80 |
| Sample 3 | 33 | 30 |
| Sample 4 | 20 | 20 |
| Sample 5 | 11 | 10 |

1. An in vitro device for measuring stool alkaline phosphatase, wherein the device comprises: a bottom panel containing a well for holding stool, an upper panel (lid) to which is attached a strip of chromatography paper impregnated with a substrate of alkaline phosphatase, the upper lid containing a hole making the chromatography paper visible.

2. The device of claim 1, wherein said measuring filling the stool well of the bottom panel with stool, making the stool surface smooth with a spatula, moistening the chromatography paper with water or a buffer, placing the upper panel (lid) on top of the bottom panel making sure the chromatography paper is in contact with the stool, waiting a specified period of time to allow the color to develop, comparing the developed color with photographs of standards of alkaline phosphatase; and quantifying the concentration of phosphatase in said stool sample.

3. The device of claim 2 or any of claims 1-2, wherein said measuring comprises: filling-in the stool well of the bottom panel with stool, making the stool surface smooth with a spatula, moistening the chromatography paper with water or a buffer, placing the upper panel (lid) on top of the bottom panel making sure the chromatography paper is in contact with the stool, waiting a specified period of time to allow the color to develop, taking photographs, quantifying the pixels of photographs, comparing the number of pixels of the photograph of the stool sample with the pixels of photographs of standards of alkaline phosphatase; and quantifying the concentration of phosphatase in said stool sample.

4. The device of claim 3 or any of claims 1-3, wherein said measuring comprises: filling-in the stool well of the bottom panel with stool, making the stool surface smooth with a spatula, moistening the chromatography paper with water or a buffer, placing the upper panel (lid) on top of the bottom panel making sure the chromatography paper is in contact with the stool, waiting a specified period of time to allow the color to develop, taking photographs, comparing the photograph of the stool sample with the photographs of standards of alkaline phosphatase, wherein such comparison is performed by a computer program for image similarity analysis; and quantifying the concentration of phosphatase in said stool sample.

5. The device of claim 4 or any of claims 1-4, wherein the upper lid can be transparent making the chromatography paper visible.

6. The device of claim 5 or any of claims 1-5, wherein said chromatography paper can be replaced with blotting paper, nylon or nitrocellulose membrane.

7. The device of claim 6 or any of claims 1-6, wherein said stool is from a human, pig, sheep, goat, cow, horse, clog, cat, monkey, rabbit, rat, mouse, chicken, turkey, or fish, preferably human.

8. The device of any one of claim 7 or any of claims 1-7, wherein said substrate for phosphatase comprises p-nitrophenyl phosphate, 5-Bromo-4-chloro-3-indolyl phosphate/nitro blue tetrazolium substrate system, Fast Red TR/Naphthol substrate system, CDP-star substrate (2-chloro-5-(4-methoxyspiro{1,2-dioxetane-3,2′(5′-chloro)-tricyclo [3.3.1.13.7]decan}-4-yl)-1-phenyl phosphate disodium salt) or combinations thereof.

9. The device of any one of claim 8 or any of claims 1-8, wherein said phosphatase comprises an alkaline phosphatase, intestinal alkaline phosphatase, placental alkaline phosphatase, nonspecific tissue alkaline phosphatase (liver/bone/kidney alkaline phosphatase), germ cell alkaline phosphatase, neutrophil alkaline phosphatase, bacterial alkaline phosphatase, an acid phosphatase or a peptide with phosphatase activity, preferably intestinal alkaline phosphatase.

10. The device of claim 9 or any of claims 1-9 can be modified, wherein the device comprises: using a strip of chromatography paper that is not impregnated with a substrate of alkaline phosphatase, an open-ended cylindrical or rectangular insert to be partly inserted in stool as such that the insert can hold liquid reagent, liquid reagent containing substrate for alkaline phosphatase is poured in the cylindrical or rectangular insert partially inserted in stool thus allowing alkaline phosphatase reaction on stool surface to continue, placing the upper panel (lid) with the chromatography paper on top of the bottom panel containing the cylindrical or rectangular insert, waiting a specified period of time, inverting the device to transfer the reagent containing phosphatase reaction product to the chromatography paper, comparing the color of the chromatography paper with photographs of standards of alkaline phosphatase, quantifying the concentration of phosphatase in said stool sample, taking photographs, counting the pixels of photographs, comparing the number of pixels of the photograph of the stool sample with the pixels of photographs of standards of alkaline phosphatase; and quantifying the concentration of phosphatase in said stool sample.

11. The device of claim 10 or any of claims 1-10 can be modified, wherein the device comprises: using a strip of chromatography paper that is not impregnated with a substrate of alkaline phosphatase, an open-ended cylindrical or rectangular insert (alternative word?) to be partly inserted in stool as such that the insert can hold liquid reagent, liquid reagent containing substrate for alkaline phosphatase is poured in the cylindrical or rectangular insert partially inserted in stool thus allowing alkaline phosphatase reaction on stool surface to continue, placing the upper panel (lid) with the chromatography paper on top of the bottom panel containing the cylindrical or rectangular insert, waiting a specified period of time, inverting the device to transfer the reagent containing phosphatase reaction product to the chromatography paper, comparing the color of the chromatography paper with photographs of standards of alkaline phosphatase, quantifying the concentration of phosphatase in said stool sample, taking photographs, comparing the photograph of the stool sample with the photographs of standards of alkaline phosphatase, wherein such comparison is performed by a computer program for image similarity analysis; and quantifying the concentration of phosphatase in said stool sample.

12. A kit containing a device of claim 11 or any of claims 1-11, for measuring stool alkaline phosphatase, wherein the kit contains any or all of the followings: a device for measuring stool alkaline phosphatase, substrate for alkaline phosphatase, buffer for alkaline phosphatase reaction, gloves, spatula, stool collection pot and information on how to perform alkaline phosphatase assay using the kit.

13. The method for measuring stool alkaline phosphatase using of a kit containing a device of claim 12 or any of claims 1-12, wherein the kit contains any or all of the followings: a device for measuring stool alkaline phosphatase, substrate for alkaline phosphatase, buffer for alkaline phosphatase reaction, gloves, spatula, stool collection pot and information on how to perform alkaline phosphatase assay using the kit.

14. The use of a kit containing a device of claim 12 or any of claims 1-12, for measuring stool alkaline phosphatase, wherein the kit contains any or all of the followings: a device for measuring stool alkaline phosphatase, substrate for alkaline phosphatase, buffer for alkaline phosphatase reaction, gloves, spatula, stool collection pot and information on how to perform alkaline phosphatase assay using the kit.
